(19) United States
(12) Patent Application Publication
(10) Pub. No.: US 2023/0380902 A1
(43) Pub. Date: Nov. 30, 2023

(54) SYSTEMS AND METHODS FOR MRI-GUIDED INTERSTITIAL THERMAL THERAPY

(71) Applicant: Desert Medical Imaging, LLC, Indian Wells, CA (US)

(72) Inventors: Bernadette M. Greenwood, Indian Wells, CA (US); John F. Feller, Indian Wells, CA (US); Wesley R. Jones, Indian Wells, CA (US)

(73) Assignee: Desert Medical Imaging, LLC, Indian Wells, CA (US)

(21) Appl. No.: 18/229,085

(22) Filed: Aug. 1, 2023

Related U.S. Application Data
(63) Continuation of application No. 15/894,190, filed on Feb. 12, 2018, now abandoned.

(51) Int. Cl. A61B 18/24; G01R 33/563; G01R 33/48; A61B 18/22; A61B 18/00; A61B 18/20; A61B 17/00; A61B 5/055

(52) U.S. Cl. CPC A61B 18/24; A61B 5/055; G01R 33/4808; A61B 18/22; A61B 2018/00904; A61B 2018/00708; A61B 2018/00547; A61B 2018/00982; A61B 2018/00678; G01R 33/4804; A61B 2018/2005; A61B 2018/206; A61B 2018/00809; A61B 2018/00791; A61B 2018/00023; A61B 2018/00577; A61B 2017/00128; G01R 33/56341

(57) ABSTRACT

A method for magnetic resonance imaging (MRI)-guided interstitial thermal therapy includes receiving MRI data for tissue of a patient, generating an apparent diffusion coefficient (ADC) map from the MRI data, identifying a target site for thermal therapy based on the ADC map, wherein the target site is a tumor identified by low ADC values on the ADC map, planning the thermal therapy for the target site including identifying a plurality of individual localized areas to be ablated during delivery of the thermal therapy, the plurality of individual localized areas having the lowest ADC values among the low ADC values within the target site, activating a laser to deliver the thermal therapy to the plurality of individual localized areas using a laser fiber, and monitoring progress of the thermal therapy using MR thermometry.

### CROSS-REFERENCE TO RELATED APPLICATIONS

This application is a continuation of U.S. patent application Ser. No. 15/894,190, filed Feb. 12, 2018, which is incorporated by reference herein in its entirety.

### BACKGROUND

The present disclosure relates generally to the field of thermal therapy for cancer treatment and, in particular, to using laser ablation therapy to treat prostate cancer.

Prostate cancer is the most frequently diagnosed non-dermatological cancer in men. Although prostate cancer mortality rates have dropped, one in six men will be diagnosed with prostate cancer in his lifetime. The two most common treatments for clinically localized prostate cancer (e.g., cancer that is localized to the prostate and has not spread) are radical prostatectomy and external beam radiation therapy. In a radical prostatectomy procedure, the prostate gland and some of the surrounding tissue is removed. With external beam radiation therapy, high-energy x-rays or other particles are focused into the prostate cancer site to kill the cancer cells. Another treatment option for prostate cancer is brachytherapy, in which radioactive implants are placed directly in the prostate to kill the cancer cells.

However, radical prostatectomy procedures are often invasive and may have lasting side effects, such as urinary incontinence, erectile dysfunction, and sterility. Likewise, men undergoing external beam radiation therapy or brachytherapy may suffer from similar side effects. Moreover, because external beam radiation therapy and brachytherapy use radiation to treat the prostate cancer, men undergoing these treatments have a small risk of developing a secondary cancer from the radiation therapy.

### SUMMARY

One embodiment relates to a method for magnetic resonance imaging (MRI)-guided interstitial thermal therapy. The method includes receiving MRI data for tissue of a patient, generating an apparent diffusion coefficient (ADC) map from the MRI data, and identifying a target site for thermal therapy based on the ADC map, wherein the target site is identified based on an area on the ADC map with a lowest ADC value. The method further includes planning the thermal therapy for the target site including identifying localized areas of the target site to be ablated first during delivery of the thermal therapy, activating a laser to deliver the thermal therapy with a laser fiber, and monitoring progress of the thermal therapy using MRI. In some arrangements, the progress of the thermal therapy is monitored using MR thermometry.

Another embodiment relates to a system for MRI-guided interstitial thermal therapy. The system includes a laser, a guide configured to direct the laser fiber to a target site for thermal therapy, and a processing circuit. The processing circuit includes a processor and a memory having instructions stored thereon that, when executed by the processor, cause the processing circuit to receive MRI data for a tissue of a patient, generate an ADC map from the MRI data, and identify the target site for the thermal therapy based on the ADC map, wherein the target site is identified based on an area on the ADC map with a lowest ADC value. The instructions further cause the processing circuit to plan the thermal therapy for the target site including identifying localized areas of the target site to be ablated first during delivery of the thermal therapy, activate the laser to deliver the thermal therapy via a laser fiber, and monitor progress of the thermal therapy using MRI. In some arrangements, the progress of the thermal therapy is monitored using MR thermometry.

Another embodiment relates to a non-transitory computer readable medium including instructions contained thereon. When executed by a processor, the instructions cause a processing circuit to receive MRI data for a tissue of a patient, generate an ADC map from the MRI data, and identify the target site for the thermal therapy based on the ADC map, wherein the target site is identified based on an area on the ADC map with a lowest ADC value. The instructions further cause a processing circuit to plan the thermal therapy for the target site including identifying localized areas of the target site to be ablated first during delivery of the thermal therapy, activate a laser to deliver the thermal therapy via a laser fiber, and monitor progress of the thermal therapy using MRI. In some arrangements, the progress of the thermal therapy is monitored using MR thermometry.

### BRIEF DESCRIPTION OF THE DRAWINGS

FIG. 1 illustrates a perspective view of a surgical system for MRI-guided interstitial thermal therapy, according to an exemplary embodiment.

FIG. 2 illustrates a side view of a transrectal guide used in the surgical system of FIG. 1, according to an exemplary embodiment.

FIG. 3 illustrates a side view of a laser system used in the surgical system of FIG. 1, according to an exemplary embodiment.

FIG. 4 illustrates a side view of a tip of the laser system of FIG. 3, according to an exemplary embodiment.

FIG. 5 illustrates a block diagram of a computing system used in the surgical system of FIG. 1, according to an exemplary embodiment.

FIG. 6 is a flow chart of a method of performing MRI-guided interstitial thermal therapy, according to an exemplary embodiment.

FIG. 7 illustrates a view of a portion of the laser system of FIG. 4 delivering thermal therapy at a target site, according to an example embodiment.

### DETAILED DESCRIPTION

The present disclosure introduces an MRI-guided interstitial thermal therapy that may be used, for example, to treat prostate cancer. With MRI-guided interstitial thermal therapy, a laser fiber is guided to the treatment site and used to ablate target cells. The progress of the thermal therapy is planned and monitored using MRI, as discussed in further detail below. As noted above, current methods for treating prostate cancer are often invasive and/or have lasting side effects, such as a urinary incontinence, erectile dysfunction, and sterility. Additionally, radiation therapies are associated with a small risk of developing a secondary cancer from the radiation. By contrast, MRI-guided interstitial therapy is targeted and minimally-invasive. The therapy may also be monitored in near real-time through MRI. Furthermore, MRI-guided interstitial therapy may be performed as an outpatient procedure. It should be understood, however, that although the present disclosure makes reference to treating prostate cancer, the systems and methods disclosed herein may be used to treat other cancers or medical conditions.

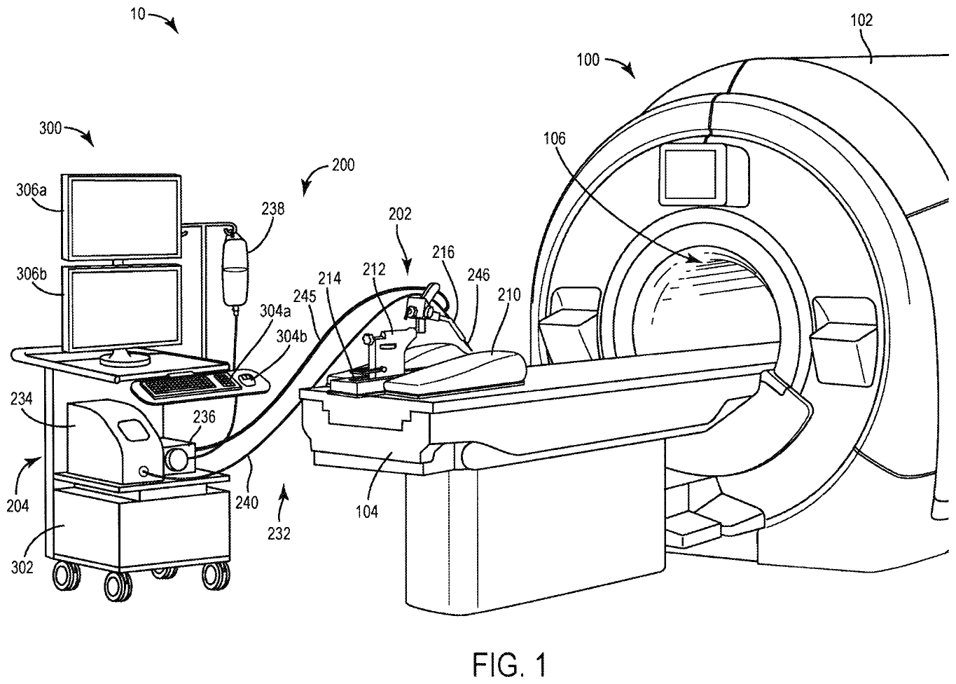

Referring now to FIG. 1, a perspective view of a surgical system 10 is shown, according to an exemplary embodiment. The surgical system 10 may be configured to perform a variety of surgical procedures, including the MRI-guided interstitial therapy described herein. As shown, the surgical system 10 includes an MRI system 100, a thermal therapy system 200, and a computing system 300.

The MRI system 100 includes an MRI scanner 102 with a scanner table 104. A patient is placed on the scanner table 104, and a portion of the patient's anatomy is moved into a scanner bore 106 of the MRI scanner 102. When in use, the MRI scanner 102 uses a magnet (not shown) to generate a magnetic field that excites hydrogen atoms in the patient anatomy being imaged that, in turn, emit radio frequency signals that are measured by a receiving coil (not shown) of the MRI scanner 102. In some embodiments, the MRI may utilize two coils. The MRI scanner 102 is connected to a computing system, such as the computing system 300 or a separate computing system, that processes the MRI data (e.g, the radio frequency signals) to create images of the patient anatomy. Additionally, to improve the contrast of MR images, the patient may be injected with a contrast agent such as gadolinium. As an illustration, with dynamic contrast-enhanced (“DCE”) imaging, the patient is injected with a contrast agent and subjected to perfusion MRI scanning to detect, for example, the flow of blood through the patient's vessels in the target tissue.

Further, the MRI system 100 may be configured to produce differently weighted images with different contrasts. As an illustration, when excited, the patient anatomy returns to an equilibrium state through T1 relaxation (associated with relaxation parallel to the magnetic field) and through T2 relaxation (associated with relaxation perpendicular to the magnetic field). As such, the image may be T1-weighted by changing the repetition time (i.e., the time between two successive excitations of the same tissue) or T2-weighted by changing the echo time (i.e., the time between an excitation pulse and the peak of the signal). As another illustration, in diffusion-weighted imaging (“DWI”), the MRI system 100 is configured to measure the random Brownian motion of water molecules within the target tissue. Apparent diffusion coefficient (“ADC”) values, which measure the magnitude of diffusion of water molecules within tissue, for the areas in the target tissue may then be calculated using the DWI data and be displayed as a parametric map. As yet another illustration, the MRI system 100 may be configured to measure the proton resonant frequency shift that occurs when water molecules in imaged tissue are heated and produce MR thermometry images (e.g., thermal maps) of the tissue. Accordingly, in some embodiments, the MRI system 100 used with MRI-guided interstitial thermal therapy described herein is configured for (a) T1 or T2-weighted axial, sagittal, and coronal imaging, (b) DWI, (c) DCE imaging, and (d) MR thermometry imaging.

Additionally, MRI systems, such as the MRI system 100, may be configured with magnets capable of generating differently sized magnetic fields. Commonly, diagnostic MRI systems are configured with a magnetic capable of generating a magnetic field of 1.5 Tesla or 3.0 Tesla. However, while an MRI system configured with a 3.0 Tesla magnet has a greater magnetic field strength than an MRI system configured with a 1.5 Tesla magnet, many artifacts are more apparent, and may be problematic, at higher field strengths. These artifacts include motion susceptibility and metallic and dielectric signal losses. Additionally, thermal maps are gradient echo-based and therefore less influenced by bowel gas, motion, and other artifact-generating problems at 1.5 Tesla. As such, in some embodiments, the MRI system 100 used with the MRI-guided interstitial thermal therapy is a 1.5 Tesla system (e.g., an MRI system configured with a 1.5 Tesla magnet or an MRI system only used with a 1.5 Tesla magnetic field).

As shown in FIG. 1, the thermal therapy system 200 includes a transrectal guide 202, a laser control system 204, and a laser system 232. The transrectal guide 202 is configured to be inserted into the rectum of a patient to guide a thermal therapy laser fiber (e.g., an ablation laser fiber) to the therapy site in the patient's prostate (e.g., the site of the prostate cancer). In some embodiments, the transrectal guide 202 is configured to guide a biopsy needle to the patient's prostate but may be repurposed to guide an ablation laser fiber to the therapy site in the patient's prostate, as described in further detail herein. For example, the transrectal guide 202 may be the DynaTRIM system sold by Invivo Corporation.

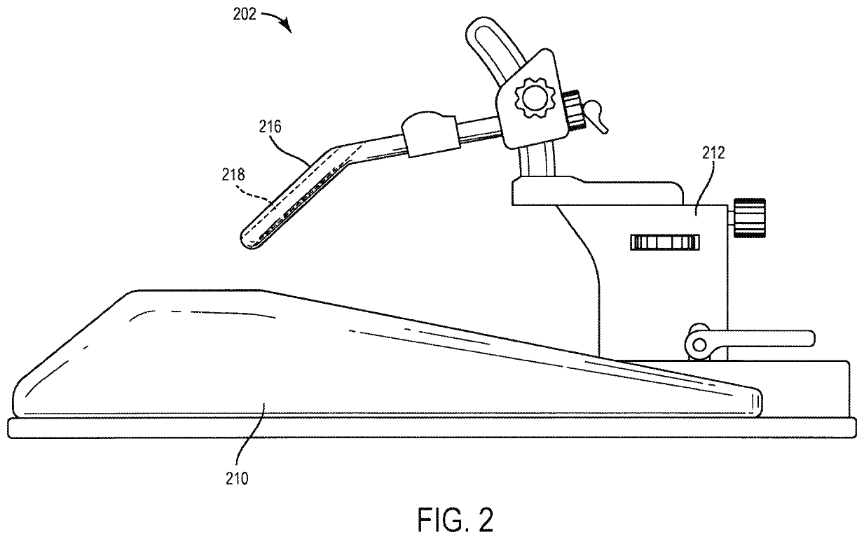

Referring now to FIG. 2, a side view of the transrectal guide 202 is shown, according to an exemplary embodiment. As shown, the transrectal guide 202 includes a base plate 210. A clamp stand 212 connects to the base plate 210 through a runner 214 (shown in FIG. 1) such that the clamp stand 212 may be pushed forward or backward on the base plate 210. The clamp stand 212 also includes multiple knobs configured to adjust the position and/or orientation of the clamp stand 212. For example, the clamp stand 212 may be configured for six-way adjustability. A needle guide 216, with a lumen 218 extending through the length of the needle guide 216, is configured to attach to the clamp stand 212 and receive a biopsy needle, a catheter, an ablation laser fiber, etc. therein. The needle guide 216 is further configured to serve as a fiducial marker when imaged using MRI (e.g., MRI imaging used for localization and planning of the ablation procedure). For example, on an MR image, the needle guide 216 appears as two bright, parallel lines. In operation, the base plate 210 rests on the MRI scanner table 104 as shown in FIG. 1. The patient lies down with his legs on the base plate 210 such that the clamp stand 212 is positioned between the patient's legs. The needle guide 216 is inserted into the patient's rectum and positioned to offer access the patient's prostate gland. The clamp stand 212 is pushed up to the needle guide 216 along the runner 214, and the position and/or orientation of the clamp stand 212 is adjusted using the knobs to align with the needle guide 216. The needle guide 216 is then coupled to the clamp stand 212 to secure the needle guide 216 in place. Once the needle guide 216 is secured, the laser fiber may be threaded through the lumen 218 of the needle guide 216 into the patient's rectum and to the target site in the patient's prostate gland.

The thermal therapy system 200 also includes the laser control system 204 and the laser system 232. The laser control system 204 (e.g., a 15 W laser control system 204 with a 980 nm diode adapted to perform ablation on target tissue) is used to power the laser system 232, which delivers the therapy. For example, the laser control system 204 and the laser system 232 may be, or include components from, the Visualase® system sold by Medtronic, Inc. As shown in FIG. 1, the laser control system 204 includes at least a laser power source 234 and a peristaltic pump 236, with the peristaltic pump 236 further connected to a saline solution bag 238.

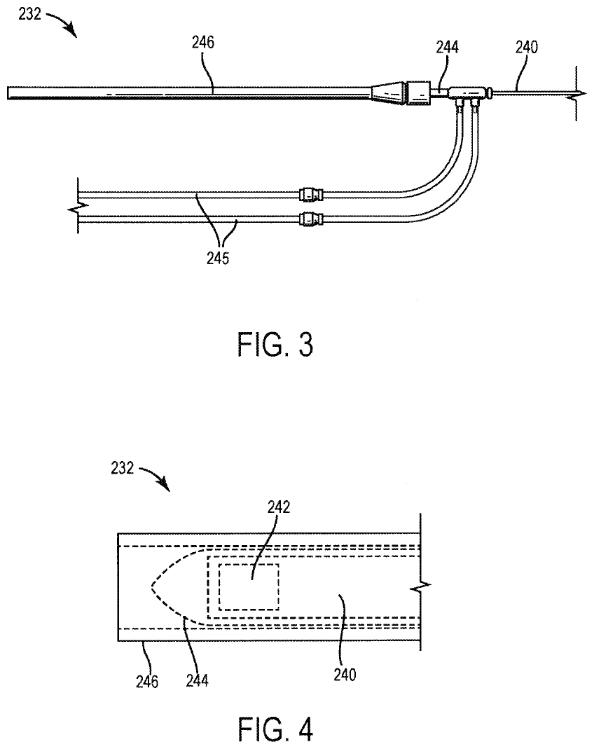

Referring now to FIG. 3, a side view of the laser system 232 is shown, according to an exemplary embodiment. To begin with, the laser system 232 includes a laser fiber 240 that is connected to the laser power source 234, as shown in FIG. 1. The laser fiber 240 is configured to thread inside a lumen of a cooling catheter 244. The cooling catheter 244 receives saline solution from the peristaltic pump 236 through cooling tubing 245, as further shown in FIG. 1. In turn, the cooling catheter 244 is configured to thread inside a lumen of a sheath 246.

In various embodiments, to position the laser fiber 240 at the target ablation area, the sheath 246 with a trochar fitted inside the lumen of the sheath 246 is threaded through the lumen 218 of the needle guide 216 to the target ablation site. The trochar is then removed and replaced with the cooling catheter 244 with a stiffener inside the catheter 244. The stiffener is removed, and the laser fiber 240 is threaded inside the catheter 244 to the target ablation site.

Referring now to FIG. 4, a side view of the distal end of the laser system 232 is shown, according to an exemplary embodiment. As shown in FIG. 4, the laser fiber 240 includes a heat-diffusing laser tip 242 configured to be heated by the laser power source 234 and thereby deliver thermal therapy (e.g., ablation therapy) to target tissues. The cooling catheter 244 (e.g., a water-cooled or saline-cooled catheter), into which the laser fiber 240 is threaded, surrounds the distal end of the laser fiber 240. The peristaltic pump 236 pumps saline solution from the saline solution bag 238 through the water-cooled catheter 244 via the cooling tubing 245 to cool the length of the laser fiber 240. As such, when the laser tip 242 is heated by the laser power source 234, the laser tip 242 ablates target tissues, but the cooling catheter 244 mitigates the risk of charring adjacent to the surface of the catheter 244, which would result in undesired heat absorption rates. Additionally, because the laser fiber 240 is threaded within the catheter 244, the laser fiber 240 can be slid to a different position within the catheter 244 to reposition the laser tip 242 for ablation in a different location.

Alternatively, in other embodiments, the laser fiber 240 is configured to distribute heat while applying thermal therapy such that cooling the laser fiber 240 with saline is unnecessary. In such embodiments, the laser control system 204 may accordingly include just the laser power source 234, and the laser system 232 may consist of the laser fiber 240 with the heat-diffusing laser tip 242 and the sheath 246. As an example, the laser control system 204 and the laser system 232 may be, or include components of, the Tranberg® system sold by Clinical Laserthermia Systems AB. Thus, when the laser system 232 is positioned at the target ablation site, the sheath 246 with the trochar is first threaded to the site, and the trochar is removed and replaced with the non-cooled laser fiber 240.

The computing system 300 includes hardware and software for operation and control of the components of the surgical system 10. As such, in various embodiments, the computing system 300 includes hardware and/or software for operation and control of the MRI system 100 and the thermal therapy system 200. However, it should be understood that while the computing system 300 is shown in FIG. 1 as a single computing system, the computing system 300 may instead include multiple computing systems configured for operation and control of specific components of the surgical system 10. For example, the computing system 300 may include a personal computer and a surgical workstation. As another example, separate computing systems may be connected to and configured to control each of the MRI system 100 and the thermal therapy system 200. Whether provided by a single computing system, or multiple, such hardware and/or software are configured to enable a user of the surgical system 10 (e.g., a practitioner) to perform the techniques described herein. As an illustration, the computing system 300 shown in FIG. 1 includes a surgical controller 302, input devices 304a (e.g., a keyboard) and 304b (e.g., a mouse), and display devices 306a and 306b (e.g., displays or monitors).

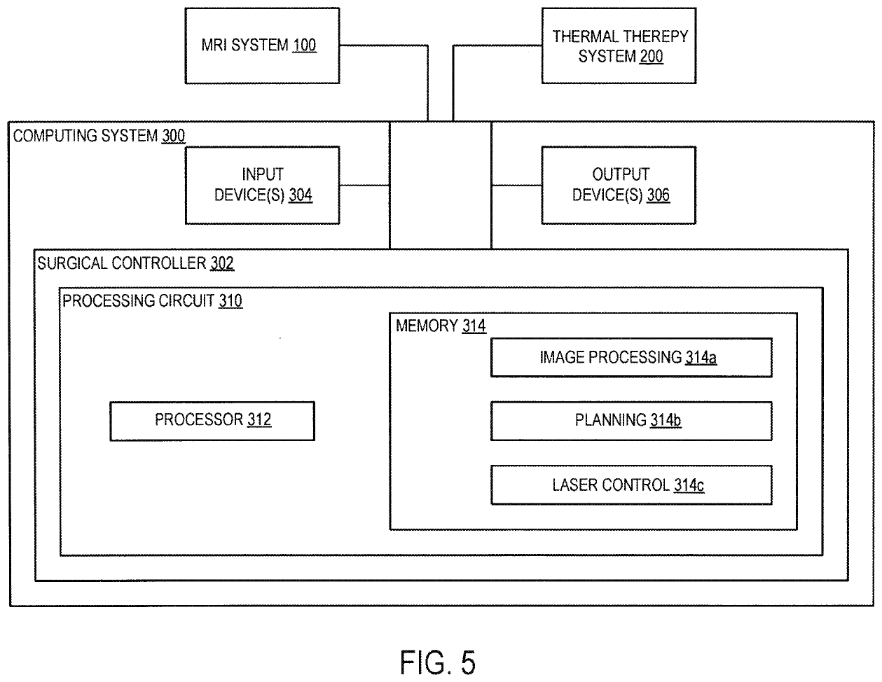

Referring now to FIG. 5, a block diagram of the computing system 300 is shown, according to an exemplary embodiment. In the embodiment illustrated in FIG. 5, the computing system 300 is connected to the MRI system 100 and the thermal therapy system 200. Again, it is to be understood that the MRI system 100 and the thermal therapy system 200 may be controlled by separate computing systems, which may carry out the processes similar to computing system 300 for its specific processes. The computing system 300 includes at least one input device 304 and at least one output device 306. Additionally, as shown in FIG. 5, the computing system 300 is a programmable, processor-based system. As such, the surgical controller 302 includes a processing circuit 310 having a processor 312 and a memory 314. The processor 312 may be implemented as a general-purpose processor executing one or more instructions (e.g., computer code or programs) to perform actions by operating on input data and generating output. Alternatively, the processes and logic flows described herein may also be performed by special purpose logic circuitry, such as a field programmable gate array (“FPGA”) or an application specific integrated circuit (“ASIC”), a group of processing components, or other suitable electronic processing components.

A processor may receive instructions, as well as input data, from a read only memory (“ROM”), a random access memory (“RAM”), or both. Accordingly, the memory 314 (e.g., memory, memory unit, storage device, etc.) comprises one or more devices (e.g., RAM, ROM, Flash-memory, hard disk storage, etc.) structured for storing instructions and/or data for completing or facilitating the various processes described in the present application. The memory 314 may be or include volatile memory or non-volatile memory. Moreover, the memory 314 may include database components, object code components, script components, or any other type of information structure for supporting the various activities described in the present application. Accordingly, in the embodiment of FIG. 5, the memory 314 is communicably connected to the processor 312 and includes instructions (e.g., computer code or programs) for executing one or more processes described herein. For example, the memory 314 may contain a variety of modules, each capable of storing instructions and/or data related to specific types of functions. Accordingly, the memory 314 contains several modules related to surgical procedures, such as an image processing module 314a, a planning module 314b, and a laser control module 314c, as shown in FIG. 5.

In various embodiments, the computing system 300 may be implemented as a system that includes a back-end component (e.g., as a data server), includes a middleware component (e.g., an application server), or includes a front-end component (e.g., a client computer having a graphical user interface (“GUI”) or a web browser through which a user may interact with an embodiment of the subject matter described in this specification), or that includes any combination of one or more such back-end, middleware, or front-end components. The components of the computing system 300 may be interconnected by any form or medium of digital data communication (e.g., a communication network). Moreover, the computing system 300 may at least partially be embedded in another device, such as a mobile telephone, a tablet, a personal digital assistant (“PDA”), a mobile audio or video player, a game console, a Global Positioning System (“GPS”) receiver, or a portable storage device (e.g., a universal serial bus (“USB”) flash drive), to name just a few. Devices suitable for storing computer program instructions and data include all forms of non-volatile memory, media, and memory devices, including by way of example semiconductor memory devices (e.g., EPROM, EEPROM, and flash memory devices), magnetic disks (e.g., internal hard disks or removable disks), magneto optical disks, and CD ROM and DVD-ROM disks. Further, the processor 312 and the memory 314 can be supplemented by, or incorporated in, special purpose logic circuitry.

Additionally, the computing system 300 includes at least one input device 304 and at least one output device 306. Input device(s) 304 enable a user to communicate with the computing system 300. For example, as shown in FIG. 1, input devices 304 may include a keyboard 304a and a mouse 304b through which a user may provide input to the surgical controller 302. However, the input device 304 may be any device enabling a user to provide an input to the surgical controller 302, such as a keyboard, a mouse, a trackball, a touchscreen, a touchpad, voice recognition hardware or software, dials, switches, buttons, a trackable probe, a foot pedal, a remote control device, a scanner, a camera, a microphone, or a joystick.

Output device(s) 306 enable the computing system 300 to provide information to a user. Such information may include images of anatomy generated from an image data set obtained using conventional imaging techniques (e.g., MRI), graphical models (e.g., CAD models of implants, instruments, anatomy, etc.), graphical representations of a tracked object (e.g., anatomy, tools, implants, etc.), constraint data (e.g., axes, articular surfaces, etc.), representations of implant components, digital or video images, registration information, calibration information, patient data, user data, measurement data, software menus, selection buttons, status information, and the like. As such, as shown in FIG. 1, output devices 306 may include displays 306a and 306b. However, the output device 306 may include any device enabling the computing system 300 to communicate with a user, such as a printer, a speaker, a vibrating component (e.g., a motor), illuminating icons, LEDs, an output device engageable to the computing system 300 via a USB, via a serial cable, via an Ethernet connection, or wirelessly, and so on.

Moreover, in some embodiments, the input device 304 may also serve as the output device 306 and accordingly be configured to gather inputs from and provide feedback to the user. As an example, the input device 304 and the output device 306 may be a touchscreen. Further, in some embodiments, the input device 304 and/or the output device 306 may be a separate computing system, such as a personal computing system that the user uses to interact with the computing system 300. For example, the user may use a personal computing system to request and receive web pages from the computing system 300.

In various embodiments, as discussed in further detail below, the computing system 300 is used to generate MR images (e.g., using the image processing module 314a), plan thermal therapy (e.g., using the planning module 314b), and control the laser control system 204 (e.g., using the laser control module 314c). With respect to generating MRI images, the computing system 300 is configured to receive MRI data (e.g., radio frequency signals) from the MRI system 100 and process the MRI data to create images of the target tissue, both for planning and real-time or near real-time (e.g., with a 5-7 s refresh rate) therapy purposes. Furthermore, the computing system 300 is configured to generate ADC parametric map from DWI data, as discussed above.

With respect to planning thermal therapy, a user may use the computing system 300 to carry out various planning processes for the thermal therapy, as described in further detail below. For example, a practitioner may user the computing system 300 to identify the target site, calculate a trajectory of the laser 232 into the target site, determine localized areas of the target site to be ablated first, and the like.

With respect to controlling the laser control system 204, the computing system 300 may be used, for example, to control the output of the laser power source 234 (e.g., the amount of power provided the timing of the provided power). Additionally, the computing system 300 may be configured to implement a temperature safety system for the laser control system 204. As an example the computing system 300 may enable a user to place safety cursors associated with warning temperatures (e.g., 90° C.) on images of the target therapy site. The computing system 300 then estimates the real-time or near real-time temperature at the cursor points (e.g., using MR thermometry) and automatically deactivates the heat-diffusing laser tip 242 if the estimated temperature exceeds the warning temperature (e.g., exceeds 90° C.). In some embodiments, the computing system 300 may allow the user to place both high-temperature safety cursors (e.g., adjacent to the laser system 232) and low-temperature safety cursors (e.g., adjacent to heat-sensitive critical structures, such as the rectal wall or the external urethral sphincter) on the image of the area targeted for therapy. Alternatively, in some embodiments, one or more of the functions of controlling the computing system 300 may instead be performed by the laser power source 234.

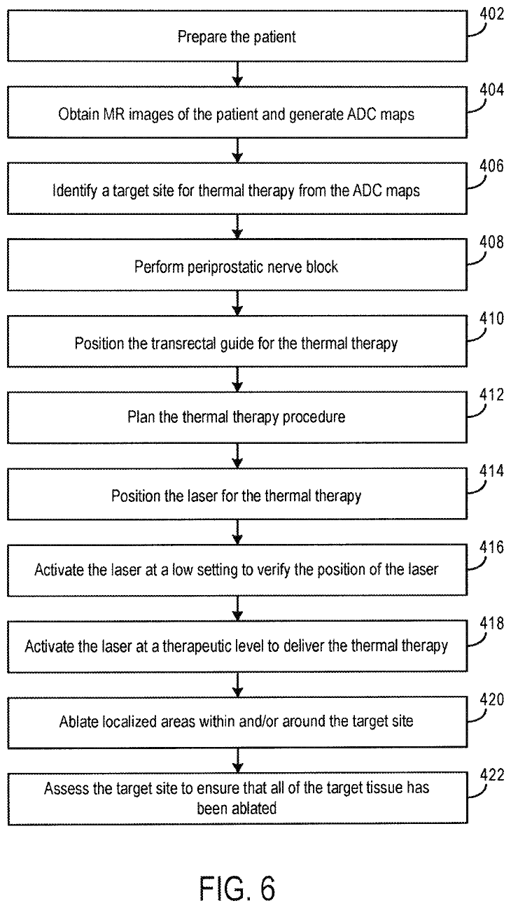

Referring now to FIG. 6, a flow chart of a method 400 of using MRI-guided interstitial thermal therapy is shown, according to an embodiment. To begin with, the patient is prepared for the procedure (step 402). For example, the patient may be given an enema followed by antibiotics. Subsequently, the patient may be instructed to lie prone on the MRI scanner table 104, with the patient's legs on the base plate 210 and on either side of the clamp stand 212 of the transrectal guide 202, and given intravenous (“IV”) sedation while on the scanner table 104.

Next, MR images of the patient's prostate tissue are obtained (step 404). As discussed above, in some embodiments, computing system 300 receives T2-weighted axial, sagittal, and coronal imaging data, DWI data, DCE imaging data, and MR thermometry data and generates MR images from the received MRI data. Additionally, the computing system 300 generates ADC maps for the patient's prostate based on the DWI data. The target site for the thermal therapy is then identified using the ADC map (step 406). In particular, prostate cancer tissue may be associated with significantly lower ADC values than surrounding noncancerous tissue. As such, a practitioner uses the ADC map to identify, or the computing system 300 automatically identifies, the area of the prostate with the lowest ADC value. In some embodiments, as the practitioner moves a cursor on a screen providing an image of the area, the ADC value at that location is shown. The ADC value is updated as the cursor is moved, and in this way, the practitioner is able to identify the lowest ADC area. This area is determined to be the target site for beginning the thermal therapy.

Once the target area is identified, a periprostatic nerve block (step 408) is performed utilizing MR guidance (e.g., based on MR images) and a 22 gauge MR-compatible needle. A volume (e.g., 10 cc) of a numbing agent such as Marcaine is bilaterally injected into the periprostatic fat at the junction between the prostate gland and the seminal vesicle. In some embodiments, 0.5% Marcaine is used, while in other embodiments, 0.25% Marcaine is used.

Next, the transrectal guide 202 is positioned for the thermal therapy (step 410). Accordingly, as described above, the needle guide 216 is inserted into the patient's rectum, for example, using a viscous lidocaine delivered transrectally as both a lubricant and an anesthetic (e.g., 11 cc of sterile 2% lidocaine jelly, or 220 mg of lidocaine HCl). In this way, the needle guide 216 is positioned to direct the laser 232 to the target site in the patient's prostate gland. Because the needle guide 216 is also configured to be an MRI fiducial marker, the position of the needle guide 216 may be verified in near real-time (e.g. with a 5-7 s delay per update) using MR images. The needle guide 216 is then fixed in place by sliding the clamp stand 212 to the needle guide 216 along the runner 214 and attaching the needle guide 216 to the clamp stand 212.

The thermal therapy procedure is then planned (step 412). Planning the thermal therapy procedure includes calculating the trajectory for the laser system 232 using the computing system 300. For example, the user may use the mouse 304b to place a cursor on the tip of the needle guide 216 and a cursor on the target site as shown on an MR image displayed on the displays 306a and/or 306b. The computing system 300 may then determine the difference between the starting position of the needle guide 216 and the target site and display (e.g., via the display 306a and/or 306b) coordinates to adjust the needle guide 216 to achieve the desired trajectory. The user may then adjust the needle guide 216 accordingly. In some examples, the position of the needle guide 216 may further be verified by inserting a needle system (e.g., a 150 nm 13-gauge MR-conditional coaxial needle system) through the needle guide 216 and obtaining a confirmation scan of the needle system using MR images. Adjustments may then be made as needed. In various embodiments, the planning process may be carried out using specialized hardware and/or software incorporated as part of the computing system 300, such as the DynaCAD processing system sold by Invivo Corporation.

Further, planning the thermal therapy procedure may include additional steps, such as placing high-temperature safety cursors and/or low-temperature safety cursors on an MR image of the target site using the computing system 300, as discussed above. As an example, the user may use the mouse 304b to place low-temperature safety cursors on tissues that are sensitive to heat, such as the rectal wall or the external urethral sphincter, and place high-temperature safety cursors on tissues that will be adjacent to the laser system 232. In various embodiments, the placement of safety cursors may be carried out using specialized hardware and/or software incorporated as part of the computing system 300, such as the Visualase system sold by Medtronic Inc.

Once the thermal therapy is planned, the laser system 232 is positioned for the thermal therapy (step 414). In some embodiments, first, the sheath 246, with a trochar inside, is inserted into the patient's rectum and guided to the target site through the rectal wall via the lumen 218 of the needle guide 216. Once the placement of the sheath 246 is verified using MRI (e.g., using a two-dimensional fast steady-state acquisition in the axial and sagittal planes), the stiffener is removed and replaced with the cooling catheter 244 with a stiffener inside. When the cooling catheter 244 is in position, the stiffener is removed and replaced with the laser fiber 240. For example, the laser fiber 240 selected for the procedure may have a 1 cm heat-diffusing tip (e.g., for smaller tumors) or a 1.5 cm heat-diffusing tip (e.g., for larger tumors). Alternatively, in other embodiments, the laser system 232 may include just the sheath 246 and the laser fiber 240 (e.g., because the laser fiber 240 does not require cooling). Accordingly, once the placement of the sheath 246 is verified, the laser fiber 240 is threaded directly through the sheath 246 to the target site.

Once the laser system 232 is positioned at the target site, the position of the laser system 232, and specifically the laser fiber 240, is then verified by activating the heat-diffusing laser tip 242 of the laser fiber 240 at a low setting (step 416). For example, the laser tip 242 is activated using a test dose of 4.5 W (i.e., at 30% power on a 15 W system). The heat from the laser tip 242 is visualized using MRI (e.g., quantitative temperature images produced via MR thermometry) to ensure that the laser fiber 240 is in the correct location before thermal therapy is started. However, the temperature remains below 43° C. and therefore will not cause damage if the laser fiber 240 is in the incorrect location. If the laser fiber 240 is in the correct location, the method 400 proceeds. If the laser fiber 240 is not in the correct location, the positions of the needle guide 216, sheath 246, cooling catheter 244, and/or laser fiber 240 are modified, and the planning performed at step 412 may be revised.

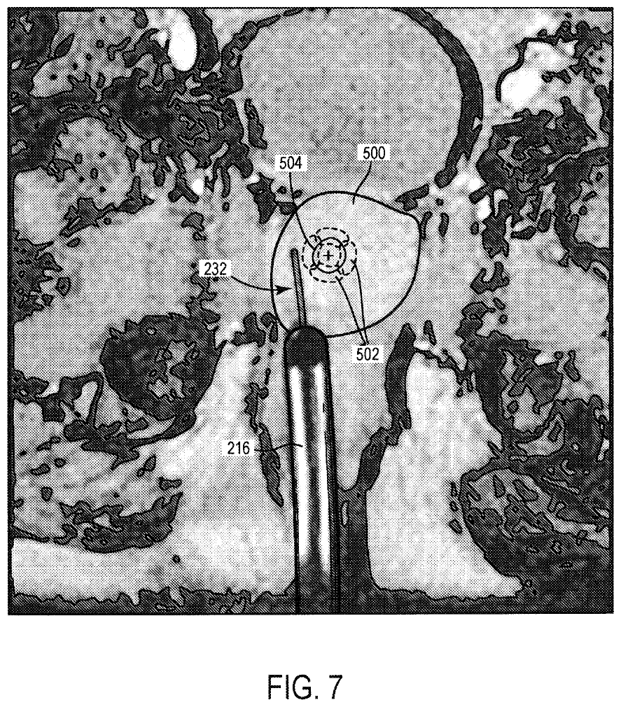

If the position of the laser fiber 240 is correct, the heat-diffusing laser tip 242 of the laser fiber 240 is activated at a therapeutic level to deliver the thermal therapy (step 418). For example, the therapeutic level for a 15 W laser fiber 240 operating at 980 nm may be 80-90% of the maximum power, or 12-13.5 W, with an exposure time of 120-150 s. Additionally, rather than applying therapy to the target site as a whole, at step 420 the laser fiber 240 is used to ablate discrete, localized areas within and/or around the target site. Referring now to FIG. 7, an example view of the needle guide 216 and laser fiber 240 delivering thermal therapy at the target site is shown, according to an exemplary embodiment. As shown in FIG. 7, the laser fiber 240 is positioned within the target site, which is shown as prostate tumor 500, and the laser fiber 240 is being used to burn localized areas 502 within the tumor 500.

The localized areas 502 to be burned may be identified during the planning process at step 412. As an illustration, during the planning process, the practitioner may use the computing system 300 to define the boundaries of the tumor and plan locations for the localized areas 502 to be within and/or on the defined boundaries. Alternatively, the localized areas 502 may be chosen by the practitioner during the procedure. In some embodiments, and as illustrated in FIG. 7, the initial localized area 502 is identified as an area having the lowest ADC value, indicated in FIG. 7 as marker 504. Additional localized areas 502 are then ablated around the area having the lowest ADC value. If necessary, the position of the needle guide 216 may also be planned and adjusted during the procedure to provide the laser 232 with access to all the selected localized areas 502. Alternatively, in other embodiments, the localized areas 502 may be located at the edges of the prostate tumor, may surround the prostate tumor 500, and/or may be spaced apart and within the tumor. Ablation of the localized areas 502 continues throughout the target area in discreet areas until all of the target area has been ablated in a “connect-the-dots” fashion. In other words, instead of ablating larger areas of tissue in a single location, several smaller localized areas 502 are ablated until the target area has been ablated.

Additionally, during the thermal therapy procedure at step 420, the computing system 300 is used to monitor the progress of the thermal therapy using MRI (e.g., using MR thermometry). In particular, a practitioner may note locations of heat-sensitive structures and monitor these structures during the ablation of the target site, or may observe estimates of irreversible damage. For example, when areas proximate to the rectal wall, external urethral sphincter, and neurovascular bundles need to be ablated, the laser fiber 240 may be used with multiple, low-energy, long-duration treatments to ablate those areas rather than at the standard therapeutic level in order to avoid irreversible damage to those tissues. Alternatively, the saline flow rate through the catheter 244 may be increased while the laser fiber 240 is being used to ablate these areas. The practitioner may manually take these measures to ensure that these heat-sensitive structures are not damaged, or the computing system 300 may automatically take these measures in response, for example, to the practitioner marking these areas with low-temperature safety cursors. Additionally, the computing system 300 may monitor the temperature of the target site (e.g., via quantitative temperature images produced using MR thermometry) to verify that the temperatures associated with the safety cursors have not been exceeded and, if they have, to automatically power down the heat-diffusing laser tip 242 of the laser fiber 240.

Once the practitioner has used the laser fiber 240 to ablate the target site, the target site is assessed to ensure that all of the target tissue (e.g., the prostate cancer tissue) has been ablated (step 422). If necessary, the laser fiber 240 is reused to ablate any remaining areas that are identified as still needing treatment. In some embodiments, the computing system 300 may further be used to plan revisions to the original thermal therapy plan based on the monitored progress of the thermal therapy in order to ensure that all of the cancer tissue has been ablated. For example, the computing system 300 may identify the areas of the target site that have been ablated (e.g., using MR thermometry) and compare the ablated areas to the target site as a whole. The computing system 300 may then display non-ablated areas to the user (e.g., via the displays 306a and/or 306b) and output coordinates for adjusting the placement of the needle guide 216 and/or the laser system 232 for ablating the remaining areas.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

Although a specific order of method steps may be described, the order of the steps may differ from what is described. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish any connection steps, processing steps, comparison steps, and decision steps.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. As described herein, embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage, other magnetic storage devices, solid state storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another, or with the two members or the two members and any additional intermediate members being attached to one another. Such joining may be permanent in nature or may be removable or releasable in nature.

The breadth of the present invention is not to be limited to the examples provided and/or the subject specification, but rather only by the scope of claim language associated with this disclosure.

What is claimed is:

1. A method for magnetic resonance imaging (MRI)-guided interstitial thermal therapy, the method comprising:
receiving MRI data for tissue of a patient;
generating an apparent diffusion coefficient (ADC) map from the MRI data;
identifying a target site for thermal therapy based on the ADC map, wherein the target site is a tumor identified by low ADC values on the ADC map;
planning the thermal therapy for the target site including identifying a plurality of individual localized areas to be ablated during delivery of the thermal therapy, the plurality of individual localized areas having the lowest ADC values among the low ADC values within the target site;
activating a laser to deliver the thermal therapy to the plurality of individual localized areas using a laser fiber; and
monitoring progress of the thermal therapy using MR thermometry.

2. The method of claim 1, wherein identifying a plurality of individual localized areas to be ablated during delivery of the thermal therapy comprises receiving, from a user, a selection of a particular position within the target area by the user's movement of a cursor, and displaying an ADC value of the tissue at the particular position, wherein the plurality of individual localized areas to be ablated during delivery of the thermal therapy are a plurality of areas having the lowest ADC value displayed to the user.

3. The method of claim 1, further comprising:
receiving, from a user, a selection of at least one location at the target site and a temperature level associated with the at least one location;
estimating a real-time temperature at the at least one selected location during the delivery of the thermal therapy; and
in response to an estimated real-time temperature exceeding the temperature level, deactivating the laser.

4. The method of claim 1, wherein planning the thermal therapy for the target site further comprises calculating a trajectory for the laser fiber based on a location of the target site and a location of a guide configured to direct the laser fiber to the target site.

5. The method of claim 4, further comprising:
calculating coordinates for adjusting the location of the guide to achieve a desired trajectory for the laser fiber; and
displaying the coordinates to a user.

6. The method of claim 1, further comprising, prior to activating the laser to deliver the thermal therapy, activating the laser at a low level to verify that the laser fiber is positioned correctly at the target site.

7. The method of claim 1, wherein the laser fiber is contained within a water-cooled catheter.

8. The method of claim 1, further comprising revising the thermal therapy plan based on the monitored progress of the thermal therapy.

9. A system for magnetic resonance imaging (MRI)-guided interstitial thermal therapy, the system comprising:
a laser fiber;
a guide configured to direct the laser fiber to a target site for thermal therapy; and
a processing circuit comprising a processor and a memory having instructions stored thereon that, when executed by the processor, cause the processing circuit to:
receive MRI data for a tissue of a patient;
generate an apparent diffusion coefficient (ADC) map from the MRI data;
identify the target site for thermal therapy based on the ADC map, wherein the target site is a tumor identified by low ADC values on the ADC map;
plan the thermal therapy for the target site including identifying a plurality of individual localized areas to be ablated during delivery of the thermal therapy, the plurality of individual localized areas having the lowest ADC values among the low ADC values within the target site;
activate the laser fiber to deliver the thermal therapy to the plurality of individual localized areas using the laser fiber; and
monitor progress of the thermal therapy using MR thermometry.

10. The system of claim 9, wherein the instructions cause the processing circuit to identify a plurality of individual localized areas to be ablated during delivery of the thermal therapy by receiving, from a user, a selection of a particular position within the target area by the user's movement of a cursor, and displaying an ADC value of the tissue at the particular position, wherein the plurality of individual localized areas to be ablated during delivery of the thermal therapy are a plurality of areas having the lowest ADC value displayed to the user.

11. The system of claim 9, wherein the instructions further cause the processing circuit to:
receive, from a user, a selection of at least one location at the target site and a temperature level associated with the at least one location;
estimate a real-time temperature at the at least one selected location during the delivery of the thermal therapy; and
in response to the estimated real-time temperature exceeding the temperature level, deactivate the laser fiber.

12. The system of claim 9, wherein the instructions cause the processing circuit to plan the thermal therapy for the target site by calculating a trajectory for the laser fiber based on a location of the target site and a location of the guide.

13. The system of claim 12, further comprising a display and wherein the instructions further cause the processing circuit to:
calculate coordinates for adjusting the location of the guide to achieve a desired trajectory for the laser fiber; and
display the coordinates to the user via the display.

14. The system of claim 9, wherein the instructions further cause the processing circuit to, prior to activating the laser fiber to deliver the thermal therapy, activate the laser fiber at a low level to verify that the laser fiber is positioned correctly at the target site.

15. The system of claim 9, wherein the system further comprises a water-cooled sheath catheter configured to receive the laser fiber.

16. The system of claim 9, wherein the instructions further cause the processing circuit to revise the thermal therapy plan based on the monitored progress of the thermal therapy.

17. A non-transitory computer readable medium including instructions container thereon that, when executed by a processor, cause a processing circuit to:
receive magnetic resonance imaging (MRI) data for a tissue of a patient;
generate an apparent diffusion coefficient (ADC) map from the MRI data;
identify the target site for thermal therapy based on the ADC map, wherein the target site is a tumor identified by low ADC values on the ADC map;
plan the thermal therapy for the target site including identifying a plurality of individual localized areas to be ablated during delivery of the thermal therapy, the plurality of individual localized areas having the lowest ADC values among the low ADC values within the target site;
activate a laser to deliver the thermal therapy to the plurality of individual localized areas using a laser fiber; and
monitor progress of the thermal therapy using MR thermometry.

18. The computer readable medium of claim 17, wherein the instructions further cause the processing circuit to:
receive, from a user, a selection of at least one location at the target site and a temperature level associated with the at least one location;
estimate a real-time temperature at the at least one selected location during the delivery of the thermal therapy; and
in response to the estimated real-time temperature exceeding the temperature level, deactivate the laser.

19. The computer readable medium of claim 17, wherein the instructions further cause the processing circuit to plan the thermal therapy for the target site by calculating a trajectory for the laser fiber based on a location of the target site and a location of the guide.

20. The computer readable medium of claim 17, wherein the instructions further cause the processing circuit to, prior to activating the laser to deliver the thermal therapy, activate the laser at a low level to verify that the laser fiber is positioned correctly at the target site.
